(19) United States
(12) Patent Application Publication
(10) Pub. No.: US 2006/0257907 A1
(43) Pub. Date: Nov. 16, 2006

(54) Packed bed for nucleic acid capture and amplification

(75) Inventors: Elizabeth K. Wheeler, Livermore, CA (US); Christopher G. Bailey, Pleasanton, CA (US); Allen T. Christian, Madison, WI (US)

Correspondence Address: Eddie E. Scott, Assistant Laboratory Counsel;Lawrence Livermore National Laboratory, P.O. Box 808, L-703, Livermore, CA, 94551, US

(73) Assignee: The Regents of the University of California

(21) Appl. No.: 11/405,128

(22) Filed: Apr. 13, 2006

Related U.S. Application Data
(60) Provisional application No. 60/673,233, filed on Apr. 19, 2005.

(51) Int. Cl. C12Q 1/68; C12P 19/34

(52) U.S. Cl. 435/6; 435/91.2

(57) ABSTRACT

A system for nucleic acid capture and amplification comprising introducing a sample potentially containing the nucleic acid into a packed bed wherein the nucleic acid adheres to the packed bed, introducing an amplification mix into the packed bed, and thermal cycling the packed bed and the nucleic acid between denaturation and annealing temperatures for PCR amplification. One embodiment provides an apparatus for DNA capture and amplification comprising a tubing or housing having a cavity, bed media in the cavity, and a heater operatively connected to the tubing or housing.

### CROSS-REFERENCE TO RELATED APPLICATIONS

This application claims the benefit of U.S. Provisional Patent Application No. 60/673,233 by Elizabeth K. Wheeler, Christopher G. Bailey, and Allen T. Christian, filed Apr. 19, 2005, and titled “FTSD (Biobriefcase flowthrough DNA cleanup and amplification chamber).” U.S. Provisional Patent Application No. 60/673,233 filed Apr. 19, 2005 and titled “FTSD (Biobriefcase flowthrough DNA cleanup and amplification chamber)” is incorporated herein by this reference.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to nucleic acid capture and amplification and more particularly to a packed bed for nucleic acid capture and amplification.

2. State of Technology

U.S. Pat. No. 5,656,493 issued Aug. 12, 1997 to Kary B. Mullis et al provides the following state of technology information: “A method, described by Saiki et al, Science, 230, 1530-1534 (1985), has been devised for amplifying one or more specific nucleic acid sequences or a mixture thereof using primers, nucleotide triphosphates, and an agent for polymerization, such as DNA polymerase. The extension product of one primer, when hybridized to the other, becomes a template for the production of the desired specific nucleic acid sequence, and vice versa. The process is repeated as often as necessary to produce the desired amount of the sequence. The method is referred to in the Science article as Polymerase Chain Reaction or ‘PCR.’”

U.S. Pat. No. 6,372,486 for a thermo cycler to David M. Fripp issued Apr. 16, 2002 provides the following state of technology information: “Traditionally, scientists have used the technique of the Polymerase Chain Reaction (PCR) to synthesize defined sequences of DNA. This generally involves a three step procedure: separation of the DNA to be amplified (template DNA); annealing of short complimentary DNA sequences (primers) to the template DNA and finally the addition of deoxynucleotides to the primer strands in order to copy the template DNA. This is usually performed in a thermal cycling machine where a cycle of three different temperatures is repeated approximately 25-35 times. Template DNA separation and synthesis steps occur at defined temperatures.”

United States Patent Application Publication No. 2002/0072112 for a thermal cycler for automatic performance of the polymerase chain reaction with close temperature control to John Atwood published Jun. 13, 2002 provides the following state of technology information, “Applications of PCR technology are now moving from basic research to applications in which large numbers of similar amplifications are routinely run. These areas include diagnostic research, biopharmaceutical development, genetic analysis, and environmental testing. Users in these areas would benefit from a high performance PCR system that would provide the user with high throughput, rapid turn-around time, and reproducible results. Users in these areas must be assured of reproducibility from sample-to-sample, run-to-run, lab-to-lab, and instrument-to-instrument.”

U.S. Pat. No. 5,935,825 issued Aug. 10, 1999 to Naoyuki Nishimura et al provides the following state of technology information: “The PCR is an In vitro method for the enzymatic synthesis of specific DNA sequences, using two oligonucleotide primers that hybridize to opposite strands and flank the region of interest in the target DNA is described in U.S. Pat. Nos. 4,683,195 and 4,683,202 by K. B. Mullis et al. . . . One disadvantage to using PCR is that impurities such as pigmentary compounds, proteins, sugars and unidentified compounds inhibit the reaction. Therefore, separation of the cells from materials and the subsequent extraction of DNA from the cells is necessary prior to amplification by PCR in order to overcome this inhibition, cellular lysis can be accomplished with enzymes, detergents or chaotropic agents and traditionally, the subsequent extraction of the nucleic acid from the cellular lysate has involved using phenol or phenol-chloroform mixture. More recent methods of purifying the DNA include the removal of impurities by using ion exchange resins, glass filter or beads or agents for protein flocculation.”

U.S. Pat. No. 5,234,809 process for isolating nucleic acid issued to Willem R. Boom et al issued Aug. 10, 1993 provided the following “a process for isolating nucleic acid from a nucleic acid-containing starting material comprising mixing the starting material, a chaotropic substance and a nucleic acid binding solid phase, separating the solid phase with the nucleic acid bound thereto from the liquid, and washing the solid phase nucleic acid complexes.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a system for nucleic acid capture and amplification. The system comprises introducing a sample potentially containing the nucleic acid into a packed bed wherein the nucleic acid adheres to the packed bed, introducing the amplification mix, typically nucleic acid mix, into the packed bed, and thermal cycling the packed bed and the nucleic acid between denaturation and annealing temperatures for Polymerase Chain Reaction (PCR) amplification. In one embodiment the present invention provides an apparatus for DNA capture and amplification comprising a tubing or housing having a cavity, bed media in the cavity, and a heater operatively connected to the tubing or housing.

Many PCR reactions require a cleanup step a priori, since the DNA to be amplified frequently contains contaminants that inhibit the enzymes necessary for PCR amplification. This is typically done using a two-step process, in which the sample containing the nucleic acid is passed over a bed containing oxides of either silicon or aluminum, in the presence of a chemical that binds nucleic acid to the silicon, and then the silicon is washed to remove the contaminants, while the DNA remains attached (for example see U.S. Pat. No. 5,234,809 issued to Boom et al). Finally, the nucleic acid is eluted using a different chemical, and is then amplified. There are at least three problems with the prior art systems: (1) the loss of nucleic acid in the cleanup process that remains bound to the solid phase, (2) the cost of the process, and (3) the speed with which the process occurs. The loss of nucleic acid in the cleanup process can approach 50%. The present invention overcomes or reduces one or more of the problems of the prior art systems.

It has been found that amplifying the nucleic acid while bound to the beads can improve the limit of detection of an assay by an order of magnitude. In the case of low copy number samples the probability of a successful reaction is dramatically increased when processed through a packed bed and amplified on the beads. A comparison between kits that elute the nucleic acid after concentration and the packed bed has been performed for nucleic acid in aqueous solutions. The limit of detection where all PCR reactions are positive is 100 pg for the commercially available kits. Using a packed bed improves this order of magnitude. Below these limits of detection successful PCR reactions occur but with decreasing probability. For example the packed bed detected 5 out of 8 replicates of 10 fg of input nucleic acid. Whereas, the kits based on eluting the DNA from the solid phase had zero positive hits at this amount of nucleic acid.

Uses of the present invention that provides a system for nucleic acid capture and amplification include pathology, forensics, detection of biological warfare agents, detection of bio-terrorism agents, infectious disease diagnostics, genetic testing, environmental testing, environmental monitoring, point-of care diagnostics, rapid sequencing, detection of biowarfare/bio-terrorism agents in the field, polymerase chain reactions, testing for DNA hybridization, isothermal reactions, nucleic acid sequence-based amplification, rolling-circle amplification, incubation for immunoassays, and other uses. The nucleic acid capture and amplification system of the present invention is designed for use with autonomous biomonitoring devices; and was specifically developed for a Biobriefcase biomonitoring device.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1A is an illustration of the flow process for performing DNA capture and amplification on the packed bed media.

FIG. 1B is an illustration of the flow process for performing DNA capture and amplification on the packed bed media shown in FIG. 1A with structural elements added.

FIG. 2 illustrates one embodiment of a packed bed DNA capture and amplification system constructed in accordance with the present invention.

FIG. 3 illustrates another embodiment of a packed bed DNA capture and amplification system constructed in accordance with the present invention.

FIG. 4A is an illustration of another embodiment of a flow process for performing DNA capture and amplification on the packed bed media.

FIG. 4B is an illustration of the flow process shown in FIG. 4A with structural elements added.

FIGS. 5, 6, and 7 illustrate another embodiment of a packed bed for DNA capture and amplification constructed in accordance with the present invention.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

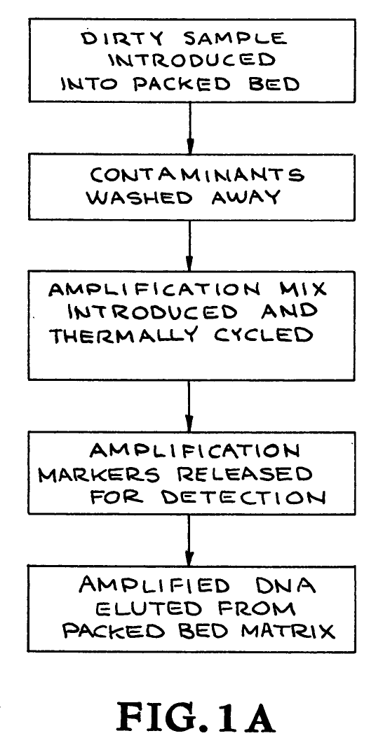

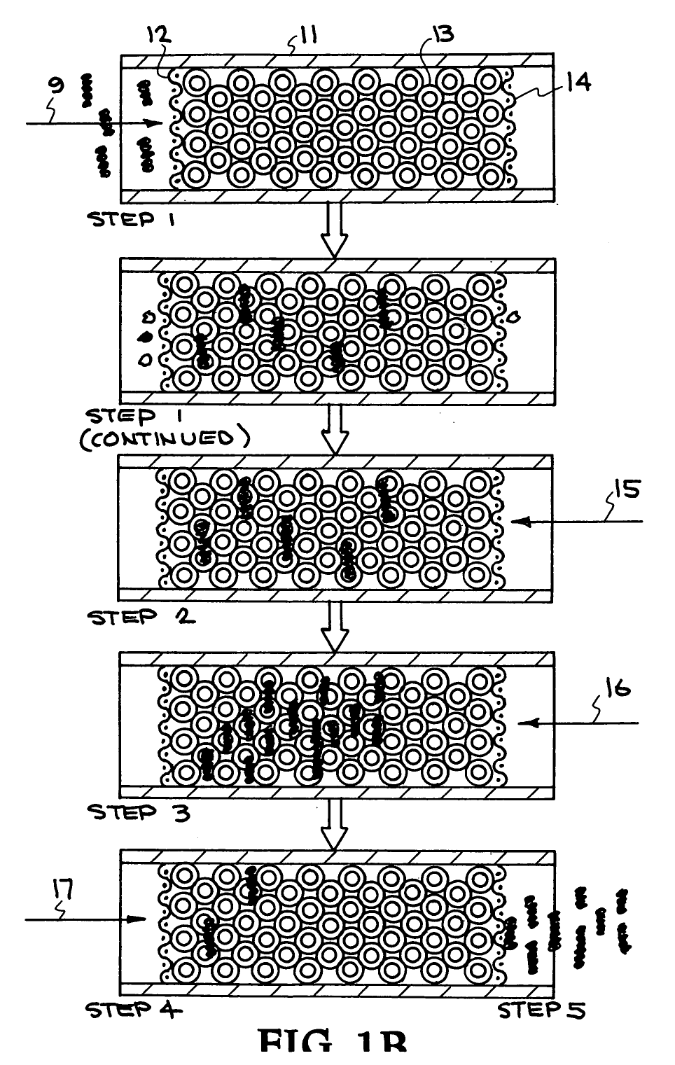

Referring now to the drawings and in particular to FIG. 1A, one embodiment of a process for packed bed for DNA capture and amplification constructed in accordance with the present invention is illustrated. Amplification of DNA is completed directly on the beads in an autonomous flow through system. The process is briefly described and summarized in FIG. 1A. The general steps illustrated in FIG. 1A occur throughout various manifestations described subsequently. This embodiment of a process for packed bed for DNA capture and amplification comprises the following steps:

Step 1, the dirty sample is introduced into the packed bed in the presence of chaotropic salt/binding agents. DNA binds to the packed bed matrix.

Step 2, contaminants are washed away.

Step 3, the amplification mix is introduced to the beads and thermally cycled.

Step 4, amplification markers are released for detection.

Step 5, amplified DNA is eluted from the packed bed matrix.

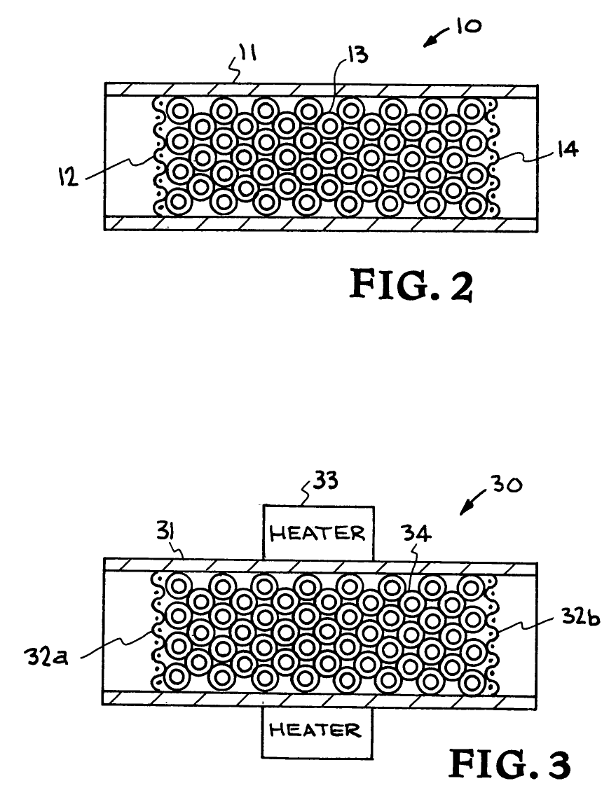

Referring again to the drawings and in particular to FIG. 2, an embodiment of a packed bed for DNA capture and amplification apparatus constructed in accordance with the present invention is illustrated. The apparatus is designated generally by the reference numeral 10. The packed bed for DNA capture and amplification apparatus 10 utilizes a biocompatible tubing or outer housing 11. The tubing or outer housing 11 is packed with bed media 13 in the form of beads.

Frits or screens 12 and 14 are use to hold the beads 13 in place. The frits or screens 12 and 14 are constructed of materials such as, but not limited to, stainless steel, plastic, other frits. The size of the frit or screen 12 and 14 is dependent on the size of beads 13 that must be maintained in the packed bed as well as the size of contaminants initially introduced. A larger frit will result in less clogging of the device. The frits 12 and 14 are inserted into the tubing 11 and secured into place. The frits or screens 12 and 14 contain the beads 13 in the tubing or outer housing 11.

Referring now to FIG. 1B, the flow process for performing DNA capture and amplification on the packed bed media of FIG. 1A is shown with structural elements of the apparatus 10 illustrated in FIG. 2 included in the illustration of the process. FIG. 1B shows the following steps and structure:

Step 1, the dirty sample 9 is introduced into the packed bed 13 in the presence of chaotropic salt/binding agents. The packed bed 13 is retained in tubing 11.

Step 1 continued, DNA binds to the packed bed matrix.

Step 2, contaminants are washed away using wash solutions 15.

Step 3, amplification mix 16 is introduced to the beads and thermally cycled.

Step 4, amplification markers 17 are released for detection.

Step 5, amplified DNA is eluted from the packed bed matrix.

The structure of a packed bed for DNA capture and amplification system constructed in accordance with the present invention having been described and illustrated in FIGS. 1A, 1B, and 2, the manufacture of the packed bed for DNA capture and amplification system 11 will now be described. An appropriate tubing 11 is selected. The tubing or outer housing 11 is constructed of materials such as, but not limited to, polypropylene, PFA, FEP, etc. The inner diameter of the tubing 11 combined with the packing media determines the volume to be amplified and analyzed.

Appropriate bed media 13 is selected. Bed media 13 comprises materials such as, but not limited to, silica beads, both regular and irregularly shaped or glass wool. The bed media 13 can be varying in size depending on tubing size to make optimized reproducible packed bed.

After selection of the tubing 11 and bed media 13 the first frit 12 is crimped into place. The bed media 13 is placed into the tubing 11. One method of getting the packed bed media into the tubing is by flowing a slurry of beads 13 in ethanol (or other solvent) into the tubing 11. The solvent is then evaporated and the second frit 14 is inserted and secured. The tubing 11 with the bed media 13 secured in place provides what is in effect a packed bed for nucleic acid capture and amplification in a thermal cycler. Thermal cyclers are known in the prior art, for example United States Patent Application Publication No. 2002/0072112 for a thermal cycler for automatic performance of the polymerase chain reaction with close temperature control to John Atwood published Jun. 13, 2002 illustrates examples of thermal cyclers. United States Patent Application Publication No. 2002/0072112 for a thermal cycler for automatic performance of the polymerase chain reaction with close temperature control to John Atwood published Jun. 13, 2002 is incorporated herein by reference.

The packed bed for DNA capture and amplification system 10 utilizes the tubing or outer housing 11 packed with bed media 13. The operation of the packed bed for DNA capture and amplification system 10 comprises a series of steps identified in FIGS. 1A and 1B as: Step 1, Step 2, Step 3, Step 4, and Step 5.

In Step 1, the dirty sample is introduced to the packed bed in the presence of chaotropic salt/binding agents. Nucleic acid adheres to the packed bed matrix.

In Step 2, contaminants are washed away.

In Step 3, the amplification mix is introduced to the packed bed/thermal chamber. By amplifying the product in situ the initial amount of DNA is increased. Whereas, if eluted before amplifying there would be some fraction \acute{η}X (where, X is the amount of DNA introduced to the system, and \acute{η} is the elution efficiency, <1 based on previous work). In situ amplification begins with X amount of DNA, (greater than \acute{η}X). The packed bed is enclosed in a thermal cycler. Thermal cycling between the denaturation and annealing temperatures is necessary for PCR amplification. These temperatures are typically, 94 and 55° C., respectively for a 2 step PCR reaction. The tubing 11 with the bed media 13 secured in place provides what is in effect a packed bed for DNA capture and amplification thermal cycler. The packed bed for DNA capture and amplification thermal cycler 10 is thermally cycled, using for example technology illustrated and described in United States Patent Application No. 2004/0072334 by William J. Benett, James, B. Richards, Paul, L. Stratton, Elizabeth, K. Wheeler, Peter Krulevitch, Steve Visuri, and John, M. Dzenitis for a Thermal Cycler published Apr. 15, 2004. United States Patent Application No. 2004/0072334 for a Thermal Cycler published Apr. 15, 2004 is incorporated herein by reference.

In Step 4, amplification markers are released for detection. For example, e-tags or taqman probes are released during amplification.

In Step 5, amplified DNA is eluted. By alternating flow directions clogging of the packed bed is minimized.

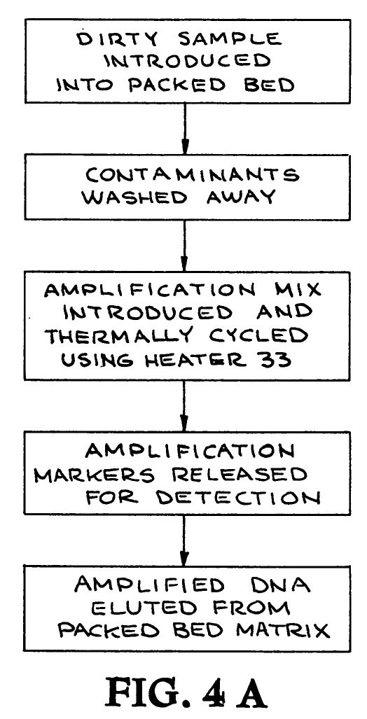

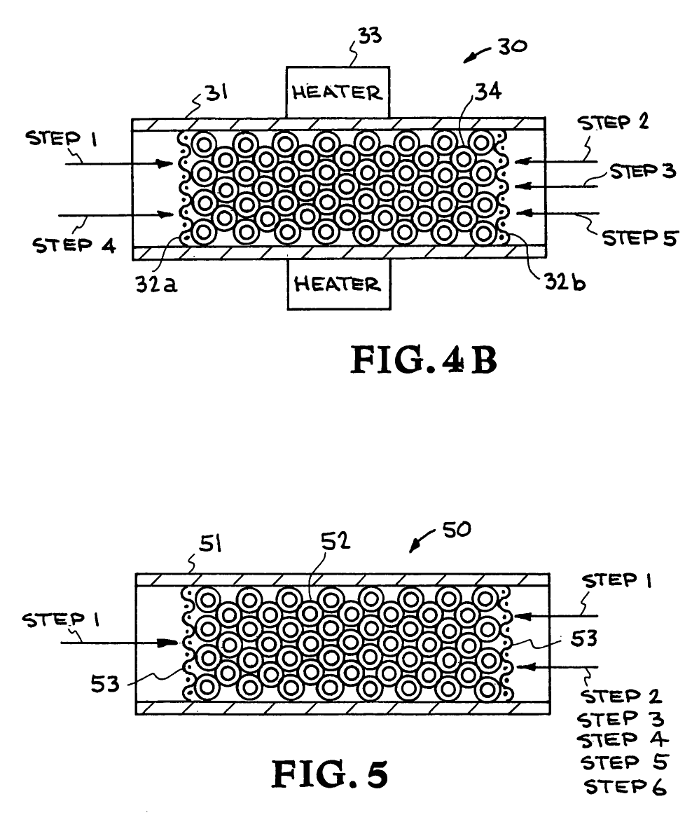

Referring now to the drawings and in particular to FIG. 4, another embodiment of a packed bed for DNA capture and amplification system constructed in accordance with the present invention is illustrated.

Uses of the nucleic acid capture and amplification system 10 include pathology, forensics, detection of biological warfare agents, detection of bio-terrorism agents, infectious disease diagnostics, genetic testing, environmental testing, environmental monitoring, point-of care diagnostics, rapid sequencing, detection of biowarfare/bio-terrorism agents in the field, polymerase chain reactions, testing for DNA hybridization, isothermal reactions, nucleic acid sequence-based amplification, rolling-circle amplification, incubation for immunoassays, and other uses. The nucleic acid capture and amplification system 10 is designed for use with autonomous biomonitoring devices; and was specifically developed for a Biobriefcase biomonitoring device.

There are many other uses for the DNA capture and amplification system 10. One is for sample preparation in law enforcement crime labs. Analysis of sexual assault samples is a laborious and time-consuming process. The forensic samples generally contain sperm cells from the perpetrator and epithelial cells from the victim. For accurate analyses, it is necessary to separate the two cell types prior to DNA analysis; DNA analysis is done on the sperm cells to determine the identity of the criminal. The present technology for doing so is fully functional, but requires skilled laboratory personnel, and considerable time. An automated device to accomplish this purpose would present considerable savings in time and expense. Another is for flow through analysis of contaminated samples, such as the PCR bacterial tests that are performed for animal care facilities. Fecal material is analyzed for the presence or absence of harmful bacteria. Currently, such tests can cost nearly $100 per sample; the DNA capture and amplification system 10 is expected to lower this by an order of magnitude by automating the cleanup and amplification procedures.

Any low copy number nucleic acid application where samples need to be purified and concentrated in an autonomous method will benefit by using this technique to capture and amplify nucleic acid within a packed bed.

Referring again to the drawings and in particular to FIG. 3, the structure of another embodiment of a packed bed for DNA capture will be described. In addition, the manufacture of the packed bed for DNA capture and amplification system and the operation of the packed bed for DNA capture and amplification system will be described. This embodiment is designated generally by the reference numeral 30. The packed bed for DNA capture and amplification system 30 utilizes a biocompatible tubing or outer housing 31. The tubing or outer housing 31 is packed with bed media 34. Bed media 34 comprises materials such as, but not limited to, silica beads, both regular and irregularly shaped. Frits or screens 32A and 32B are used to hold the bed media 34 in place.

A heating component 33 is located around the tubing 31. The heating component 33 comprises a precision resistor. The resistor 33 provides heating of the packed bed for DNA capture and amplification system 30. Temperature control is provided by sensor and control elements. The sensor and control elements provide temperature control and sensing by sensing some change in a physical characteristic. Various types of sensor and control elements are available. For example, thermocouples, resistive temperature devices (RTDs and thermistors), infrared radiators, bimetallic devices, liquid expansion devices, and change-of-state devices are available. The sensor and control element can be commercially available unit that may be obtained from OMEGA Engineering, Inc., One Omega Drive, Stamford, Conn. 06907-0047 or IMI Scott Limited, Dallimore Road, Roundthorn Industrial Estate, Wythenshawe, Manchester M23 9WJ, England.

The tubing 31 with the bed media 34 secured in place and heating component 33 provide what is in effect a packed bed for DNA capture and amplification thermal cycler. Thermal cyclers are know in the prior art, for example United States Patent Application Publication No. 2002/0072112 for a thermal cycler for automatic performance of the polymerase chain reaction with close temperature control to John Atwood published Jun. 13, 2002 illustrates examples of thermal cyclers. United States Patent Application Publication No. 2002/0072112 for a thermal cycler for automatic performance of the polymerase chain reaction with close temperature control to John Atwood published Jun. 13, 2002 is incorporated herein by reference.

The structure of a packed bed for DNA capture and amplification system 30 having been described and illustrated, the operation of the packed bed for DNA capture and amplification system 30 will now be described. The packed bed for DNA capture and amplification system 30 utilizes the tubing or outer housing 31 packed with bed media 34 surrounded by the heating unit 33. As illustrated in FIGS. 4A and 4B, the operation of the packed bed for DNA capture and amplification system 30 comprises a series of steps identified in FIGS. 4A and 4B as: Step 1, Step 2, Step 3, Step 4, and Step 5.

In Step 1, the dirty sample is introduced to the packed bed in the presence of chaotropic salt/binding agents. DNA adheres to the packed bed matrix.

In Step 2, contaminants are washed away.

In Step 3, a PCR mix is introduced to the packed bed/thermal chamber. By amplifying the product in situ the initial amount of DNA is increased. Whereas, if eluted before amplifying there would be some fraction \acute{η}X (where, X is the amount of DNA introduced to the system, and \acute{η} is the elution efficiency, <1 based on previous work). In situ amplification begins with X amount of DNA, (greater than \acute{η}X). The packed bed is enclosed in a thermal cycler. Thermal cycling between the denaturation and annealing temperatures is necessary for PCR amplification. These temperatures are typically, 94 and 55° C., respectively. The tubing 31 with the bed media 34 and heating unit 33 secured in place provide what is in effect a packed bed for DNA capture and amplification thermal cycler. The packed bed for DNA capture and amplification thermal cycler 30 is thermally cycled, using for example technology illustrated and described in United States Patent Application No. 2004/0072334 by William J. Benett, James, B. Richards, Paul, L. Stratton, Elizabeth, K. Wheeler, Peter Krulevitch, Steve Visuri, and John, M. Dzenitis for a Thermal Cycler published Apr. 15, 2004. United States Patent Application No. 2004/0072334 for a Thermal Cycler published Apr. 15, 2004 is incorporated herein by reference.

In Step 4, amplification markers are released for detection. For example, e-tags are released during amplification.

In Step 5, amplified DNA is eluted. By alternating flow directions clogging of the packed bed is minimized.

The system 10 illustrated in FIG. 2 was designed specifically for the Biobriefcase project it utilizes the flow through thermal cycler similar to those reported in US Patent Application No. 2004/0072334. However, if this is not available, amplification of the DNA on the beads in a benchtop thermal cycler is still highly advantageous in many low copy number DNA applications. FIGS. 5, 6, and 7 describe this system.

Referring to FIGS. 5, 6, and 7, the structure of a packed bed for DNA capture and amplification system and the manufacture of the packed bed for DNA capture and amplification system will be described and illustrated. The system is designated generally by the reference numeral 50. Also, the operation of the packed bed for DNA capture and amplification system 50 in conjuncture with standard benchtop equipment will be described.

The packed bed for DNA capture and amplification system 50 utilizes a tubing or outer housing 51 packed with bed media in the form of beads 52. Frits or screens 53 are use to hold the beads 52 in place. The operation of the packed bed for DNA capture and amplification system 50 comprises a series of steps identified in FIGS. 5, 6, and 7 as: Step 1, Step 2, Step 3, Step 4, Step 5, and Step 6.

In Step 1, the dirty sample is introduced to the packed bed in the presence of chaotropic salt/binding agents. DNA adheres to the packed bed matrix.

In Step 2, contaminants are washed away.

In Step 3, the beads with DNA attached, are flowed out of the packed bed in the presence of ethanol or other liquid. For dirty samples rich in particulate two frits will still be required for backflushing the system to remove any clogging of the frits. For cleaner samples, only the downstream frit need be used. If two frits are used, one frit needs to be removed prior to retrieving the beads. One illustration of this would be to simply cut the casing/tubing 11 prior to flowing the beads out of the packed bed.

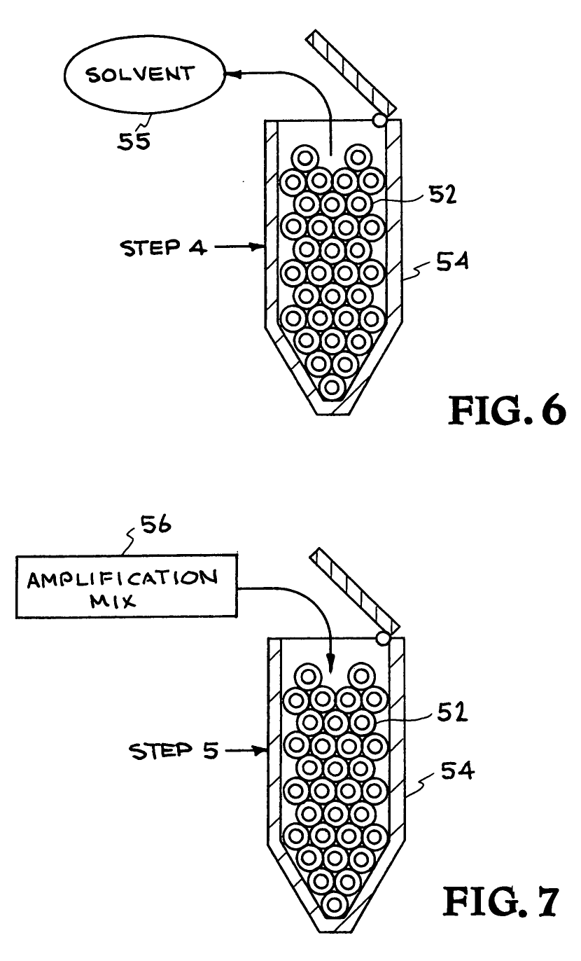

In Step 4, the beads 52 are collected in a standard PCR tube 54. The solvent 55 used to remove the beads from the packed bed housing is evaporated off. This is illustrated in FIG. 6.

In Step 5, amplification mix 56 is added to the beads 52. This is illustrated in FIG. 7.

In Step 6, the tube containing beads, DNA and amplification mix are placed into a standard benchtop thermal cycler for amplification and subsequent detection.

The nucleic acid capture and amplification system can be applied to both DNA and RNA containing samples.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A method of nucleic acid capture and amplification, comprising the steps of:
introducing a sample potentially containing the nucleic acid into a packed bed wherein the nucleic acid adheres to the packed bed,
introducing a nucleic acid mix into said packed bed, and
thermal cycling said packed bed and the nucleic acid between denaturation and annealing temperatures for polymerase chain reaction amplification.

2. The method of nucleic acid capture and amplification of claim 1 wherein said step of introducing a sample potentially containing the nucleic acid into a packed bed comprises introducing said sample into said packed bed in the presence of chaotropic salt binding agents.

3. The method of nucleic acid capture and amplification of claim 1 wherein said step of thermal cycling said packed bed and the nucleic acid between denaturation, annealing, and extension temperatures for polymerase chain reaction amplification comprises thermal cycling said packed bed and the nucleic acid between 94° C., 55° C., and 72° C.

4. The method of nucleic acid capture and amplification of claim 1 including a step of washing said sample and said packed bed.

5. The method of nucleic acid capture and amplification of claim 1 including releasing amplification markers into said packed bed.

6. The method of nucleic acid capture and amplification of claim 1 including releasing e-tags into said packed bed.

7. A method of DNA capture and amplification, comprising the steps of:
packing bed media into a tubing or housing to form a packed bed,
introducing a sample potentially containing the DNA into said packed bed wherein the DNA adheres to said bed media,
introducing a PCR mix into said packed bed, and
thermal cycling said packed bed and the DNA between denaturation and annealing temperatures for PCR amplification.

8. The method of DNA capture and amplification of claim 7 wherein said step of introducing a sample potentially containing the DNA into a packed bed comprises introducing said sample into said packed bed in the presence of chaotropic salt binding agents.

9. The method of DNA capture and amplification of claim 7 wherein said step of thermal cycling said packed bed and the DNA between denaturation and annealing temperatures for PCR amplification comprises thermal cycling said packed bed and the DNA between 94° C. and 55° C.

10. The method of DNA capture and amplification of claim 7 including releasing amplification markers into said packed bed.

11. A packed bed for DNA capture and amplification apparatus, comprising:
a tubing or housing having a cavity,
bed media in said cavity, and
a heater operatively connected to said tubing or housing.

12. The packed bed for DNA capture and amplification apparatus of claim 11, wherein said bed media comprises beads.

13. The packed bed for DNA capture and amplification apparatus of claim 11, wherein said bed media comprises regular shaped silica beads.

14. The packed bed for DNA capture and amplification apparatus of claim 11, wherein said bed media comprises irregularly shaped silica beads.

15. The packed bed for DNA capture and amplification apparatus of claim 11, wherein said bed media comprises regular shaped silica beads and irregularly shaped silica beads.

16. The packed bed for DNA capture and amplification apparatus of claim 11, including frits for holding said bed media in said tubing or housing.

17. The packed bed for DNA capture and amplification apparatus of claim 11, including screen for holding said bed media in said tubing or housing.

18. The packed bed for DNA capture and amplification apparatus of claim 11, wherein said heater comprises a resistor.

19. The packed bed for DNA capture and amplification apparatus of claim 11, wherein said heater comprises a precision resistor.

20. The packed bed for DNA capture and amplification apparatus of claim 11, wherein said heater comprises a precision resistor and control elements.
